(19) United States
(12) Patent Application Publication
(10) Pub. No.: US 2007/0149479 A1
(43) Pub. Date: Jun. 28, 2007

(54) Nanoparticulate inclusion and charge complex for pharmaceutical formulations

(76) Inventors: Katrin Claudia Fischer, Berlin (DE); Sascha General, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/514,323

(22) Filed: Sep. 1, 2006

Related U.S. Application Data
(60) Provisional application No. 60/713,332, filed on Sep. 2, 2005.

(30) Foreign Application Priority Data
Sep. 2, 2005 (DE) 102005041860.0

(51) Int. Cl. A61K 31/724; C08B 30/18

(52) U.S. Cl. 514/58; 536/46; 977/906

(57) ABSTRACT

A Nanoparticulate inclusion and charge complex that comprises at least two complex partners, whereby a complex partner is an anionic inclusion-forming agent and another complex partner is a cationic active ingredient.

This application claims the benefit of the filing date of U.S. Provisional Application Ser. No. 60/713,332 filed Sep. 2, 2005 and German Patent Application Serial No. 102005041860.0 filed Sep. 2, 2005.

This invention relates to a nanoparticulate inclusion and charge complex that comprises an anionic inclusion-forming agent and a cationic active ingredient. In more detail, this invention relates to a complex that consists of anionic beta-cyclodextrin phosphate and a (weakly) basic active ingredient in the protonated state. This invention also relates to a nanoparticle that comprises an inclusion and charge complex. In addition, this invention relates to a process for the production and use of the nanoparticle.

### BACKGROUND OF THE INVENTION

Nanoparticulate formulations as Drug Delivery Systems are described for a number of therapeutic agents and diagnostic agents in the literature and are already established as market products. By using passive and active “targeting” effects, pharmaceutical active ingredients can be brought specifically to their site of action, by which toxicity and incompatibility are prevented. Such systems also offer the possibility of improved solubility of active ingredients.

Active ingredients for a number of therapeutic applications are to be categorized based on their chemical structure as (weakly) basic pharmaceutical substances (also referred to here as pharmaceutical substance bases). Incorporation of these pharmaceutical substance bases into a particulate formulation offers decisive advantages for the therapy of inflammatory diseases (such as arthrosis) or carcinoses. Because of the altered porous tissue structure, particulate formulations are suitable for concentrating locally there by passive targeting.

A portion of the pharmaceutical substance bases is available as hydrochloride, with which good water solubility is connected in certain cases. The latter hampers incorporation into a colloidal carrier system that is usually based on polymers, however, and it thus makes difficult the use of the advantageous properties of this system, such as, for example, EPR effects (Enhanced Permeation and Retention), mucoadhesiveness in the gastrointestinal tract, size-related resorption effects, i.e. The technological difficulty consists in efficiently encapsulating a very readily water-soluble component and achieving a suitable release behavior. The reason for this is that the hydrophilic components that are to be encapsulated show the strong tendency to disperse in the production of particles in the external aqueous phase, by which only small amounts are encapsulated. To this is added the increased build-up in the outer shell of the particle, by which under certain circumstances, a large portion of the encapsulated substance is freed by “burst” effects even before reaching the site of action. The portion that is encapsulated in the core can in turn be released after polymer degradation only after a long delay.

Other pharmaceutical substance bases, which can be obtained, e.g., as salts of fumaric acid or succinic acid, show a very strong pH-dependent solution behavior. Often in the case of these active ingredients with acidic pH values (pH of 1 to 3), an acceptable or even good solubility is present that is drastically reduced along the resorption window of a pH of 4.4-7.5 in the gastrointestinal tract. An uncontrolled dropping of the free pharmaceutical substance base into this pH range is the result. Since, however, the site of the pharmaceutical substance resorption is mainly the small intestine, enormous problems occur in these pharmaceutical substances, such as, for example, an active ingredient concentration that is inadequately high for the resorption or else a resorption behavior that differs greatly interindividually, which is based on interindividual differences of the pH values prevailing in the gastrointestinal tract.

An active ingredient whose solubility shows an extreme dependence on the pH value is the phthalazine derivative (4-chlorophenyl)-[4-(4-pyridylmethyl)-phthalazin-1-yl)], whereby the succinic acid is also referred to as “vatalanib succinate” or “pynasunate.”¹ While the solubility at very low pH values, i.e., a pH of 1.0 to 2.0, is acceptable, it considerably decreases with an increasing pH. Since the resorption takes place in the small intestine, in which a pH of >5 usually prevails, it is therefore important that a sufficiently large portion of the active ingredient is present in dissolved form in this pH range and thus is available for resorption. (4-Chlorophenyl)-[4-(4-pyridylmethyl)-phthalazin-1-yl)] is an inhibitor of the three kinases of the VEGF (Vascular Endothelial Growth Factor) receptor and thus an active ingredient that now is of great interest in connection with the treatment of tumors.

The dependence of the solubility of vatalanib succinate on the pH and the temperature is listed in the following table.

Solubility (mg/ml)

| pH | Buffer | 37° C. | 20° C. |
|---|---|---|---|
| 1.0 | No | 108 | |
| 1.1 | Yes | 83 | |
| 2.0 | No | 146 | |
| 3.0 | Yes | 7.9 | |
| 3.1 | Yes | | 7.2 |
| 3.6 | No | 0.35 | |
| 3.7 | No | | 0.34 |
| 4.5 | Yes | 0.02 | |
| 5.0 | Yes | 3.7 × 10⁻³ | 2.9 × 10⁻³ |
| 7.0 | Yes | 7.1 × 10⁻⁴ | 3.1 × 10⁻⁴ |

Time and again, attempts were made to overcome formulation and application difficulties, such as, for example, a low loading quality of colloidal systems and poor water solubility. In the case of poorly soluble polymorphic substances, the use of a crystalline form with higher energy is possible, which can result in an elevated rate of solution. In practice, this often cannot be reacted, however. To this is added the most often quick conversion to more energetically advantageous forms or other salts that occurs under physiological conditions, which in turn can result in precipitation. Lahr et al., Kanikanti et al., Nakamichi et al.^{2,3,4} attempted to solve this problem by the production of amorphous dispersions with use of polymers. The conclusion here was that in some cases, there were changes in the active ingredient as well as its instability under the production conditions. The use of organic solvent as well as a very expensive and time-consuming process are additional negative effects.

Cyclodextrins and their derivatives are a class of substances that is successfully used for oral or parenteral formulation of poorly soluble pharmaceutical substance bases. Cyclodextrins are produced by the cyclizing enzymatic degradation of starch. In this connection, in formal terms a coil from the starch helix is cut out enzymatically, and the ends are newly linked. In this way, an “inner space” is produced in the cyclodextrin, in which a “guest molecule,” e.g., an active ingredient or active ingredient complex, can be incorporated (“molecular encapsulation”). By the formation of an inclusion complex in the hydrophobic interior space of the cyclodextrin, an increased solubility of, e.g., sparingly water-soluble pharmaceutical substance bases is achieved. This in turn results in a faster rate of solution and can contribute to an increase in bioavailability. Cyclodextrins and their derivatives thus represent a group of pharmaceutical adjuvants that are used as solubilizers.

As additional effects, an improved chemical and physical stability can be added. The dissociation or binding constant⁵ is decisive for the stability of the complex. The stronger the hydrophobic interactions between the guest molecule and the interior space of the cyclodextrin are, the more stable the inclusion complex is. The result is a strong increase in solubility. In the case of excessive stability of the inclusion complex, however, too little of the incorporated active ingredient is then released by dissociation. The result is nevertheless that a very little of the pharmaceutical substance is freely available, thus there is a limited bioavailability, although the solubility thereof is improved.

The “bioavailability” is a measurement variable for the proportion, in percentage, of an active ingredient of a pharmaceutical agent dose, which is available unchanged in the systemic circuit. There is thus a parameter for how quickly and to what extent a pharmaceutical agent is resorbed and is available on the site of action. In the case of medications that are administered intravenously, the bioavailability according to the definition is 100%. An absolute bioavailability is distinguished that indicates the bioavailability of a substance that is taken up in comparison to the intravenous administration, and a relative bioavailability that compares one dispensing form to another dispensing form.

Under certain structural requirements, a complexing between cyclodextrin and the guest molecule (active ingredient) to be included is possible only under drastic conditions and very incompletely. In this case, the active ingredient, because of a low binding constant, is quickly released from the complex, but the possible premature precipitation of these substances, e.g., by temperature fluctuations, is disadvantageous. A reliable use of this type of complexing for the development of a formulation is not ensured.

Under the various cyclodextrin derivatives, in particular beta-cyclodextrin is used in pharmaceutical preparations, e.g., in oral formulations as solubilizers, for stabilizing vitamin preparations or as odor and flavoring correctives.⁶ The derivative hydroxypropyl-beta-cyclodextrin is already approved as an adjuvant in an infusion solution (Sempera®). To use the advantageous pharmaceutical properties of the cyclodextrins in particulate formulations, cationically modified cyclodextrins were also described that represent alternatives in the area of gene transfection.^{7,8,9,10} Also, the use of sulfoalkyl ether-cyclodextrins is known.¹¹

In addition, the literature reports on the incorporation of active ingredient-cyclodextrin complexes in standard polymer nanoparticles, whereby the main purpose is to overcome the poor solution properties of the active ingredient after parenteral administration or after oral administration in the physiological medium.¹² The incorporation of cyclodextrin-inclusion complexes in SLNs (Solid Liquid Nanoparticle) produced a higher concentration capacity with the active ingredient, which, however, was generally always very low, in comparison to freely encapsulated hydrocortisone. In addition, an essentially smaller release of hydrocortisone form the cyclodextrin complex in comparison to pure hydrocortisone, encapsulated in SLNs, was described.¹³

Despite the previously achieved improvements, in particular basic active ingredients with low water solubility in the pharmaceutical formulation, for example cyclodextrin solutions, amorphous dispersions and colloidal transport systems (polymer nanoparticles, liposomes, SLNs, i.e.), always still have various disadvantages.

Consequently, in addition, there is also a need for pharmaceutical formulations with improved properties relative to solubility and bioavailability of the active ingredients that are contained. In this connection, it is important that the properties of the pharmaceutical substance not be improved at the expense of its stability and is not to be achieved by means of harmful adjuvants. Also, there should be practicable production methods to make possible a production that is reasonable in terms of time and cost.

It was therefore an object of the invention to make available an improved pharmaceutical formulation, which has superior properties in particular relative to the solubility and bioavailability of the active ingredients that are contained.

### SHORT VERSION OF THE INVENTION

The object of this invention is achieved by a nanoparticulate inclusion and charge complex, comprising at least two complex partners, whereby one complex partner is an anionic inclusion-forming agent and another complex partner is a cationic active ingredient.

In a preferred embodiment, the cationic active ingredient is a basic active ingredient.

In an especially preferred embodiment, the basic active ingredient is in the protonated state.

In one embodiment, the cationic active ingredient is a low-molecular active ingredient.

In one embodiment, the inclusion-forming agent is an anionically modified cyclodextrin.

In a preferred embodiment, the anionically modified cyclodextrin is a cyclodextrin phosphate, cyclodextrin sulfate, cyclodextrin carboxylate or cyclodextrin succinate.

In an especially preferred embodiment, the anionically modified cyclodextrin is a beta-cyclodextrin phosphate.

In another especially preferred embodiment, the anionically modified cyclodextrin is heptakis-(2,3-dimethyl-6-sulfato)-beta-cyclodextrin or heptakis-(2,6-diacetyl-6-sulfato)-beta-cyclodextrin.

In a preferred embodiment, the active ingredient is selected from the group that consists of pynalin, vatalanib succinate, imipramine, apomorphine, atropine, scopolamine, bamipine, astemizole, diphenhydramine, quinidine, quinine, chloroquine, chlorpromazine, chlorprothixene, codeine, ephedrine, naphazoline, oxedrine, isoprenaline, salbutamol, fenoterol, hydromorphone, hydrocodone, morphine, haloperidol, imipramine, lidocaine, loperamide, methadone, levomethadone, metoclopramide, cimetidine, naphazoline, perazine, pethidine, procaine, benzocaine, lidocaine, mepivacaine, promazine, chlorpromazine, propanolol, scopolamine, perazine, thioridazine, trimethoprim, bromhexine, clotrimazole, nitroflurantoin, diazepam, oxazepam, nitrazepam, diphenhydramine, haloperidol, imipramine, isoniazid, loperamide, metronidazole, nicotinamide, papaverine, pethidine, phenazone, ambroxol, bamipine, diphenhydramine, bromocriptine, clonidine, propanolol, metoprolol, phentolamine, sulfaguanidine, ergotamine, verapamil, diltiazem, neostigmine bromide, pilocarpine, physostigmine, ketotifen, thiamin, pyridoxine, imiquimod, irinotecan, raloxifene, tirofiban, mercaptamine bitartrate, brimonidine, tolterodine, mizolastine, abacavir, zaleplon, emedastine, amisulpride, sibutramine, levacetylmethadol, rizatriptan, lercandipine, rosiglitazon, buproprion, quetiapin, brinzolamide, lomefloxacin, almotriptan, galanthamine, desloratadine, levocetirizine, levodropropizine, oxaprozin, voriconazole, tiotropium bromide, ziprasidone, ebastine, eletriptan, imantinib, gatifloxacin, olmesartan, frovatriptan, solifenacin, manidipine, epinastine, olopatadine, escitalopram, duloxetine, a therapeutically active protein and a therapeutically active peptide and salts thereof.

In an especially preferred embodiment, [it] is the low-molecular basic vatalanib succinate.

In one embodiment, the complex is meta-stable.

In one embodiment, the complex is dissociated from inclusion-forming agents and active ingredients in the presence of an additional charged compound or another salt.

In a preferred embodiment, the additional charged compound or the salt is contained endogenically in the gastrointestinal tract and/or is fed exogenically.

In a preferred embodiment, the inclusion-forming agent and the additional charged compound or the salt go into a complex, and the dissociated active ingredient diffuses.

In a preferred embodiment, in the range of pH 4 to pH 9, the stability of the complex is independent of pH.

In an alternative preferred embodiment, in the range of pH 5 to pH 7.5, the stability of the complex is independent of pH.

In a preferred embodiment, the complex in a simulated intestinal fluid, selected from FaSSIF (Fasted State Simulated Intestinal Fluid) and FeSSIF (Fed State Simulated Intestinal Fluid), is stable.

In addition, the object of the invention is achieved by a nanoparticle, comprising an inclusion and charge complex according to this invention.

In one embodiment, the nanoparticle comprises a surface that modifies the inclusion and charge complex.

In one embodiment, the nanoparticle has a size in the range of 10 mm to 1.2 μm.

In one preferred embodiment, the nanoparticle has a size in the range of 10 nm to 500 nm.

In an especially preferred embodiment, the nanoparticle has a size in the range of 10 nm to 300 nm.

In one embodiment, the surface of the nanoparticle has a negative surface potential in the range of −10 mV to −70 mV.

In a preferred embodiment, the surface of the nanoparticle has a negative surface potential in the range of −20 mV to −60 mV.

In one embodiment, the nanoparticle comprises at least one surface-modifying compound.

In one preferred embodiment, the surface-modifying compound is covalently- or non-covalently-bonded to the surface of the nanoparticle.

In one preferred embodiment, the compound that modifies the surface has a charge that is opposite to the charge of the surface of the nanoparticle.

In an especially preferred embodiment, the surface-modifying compound is a positively charged compound.

In one preferred embodiment, the positively charged compound is a block-co-polymer.

In one especially preferred embodiment, the block-co-polymer is a cationically modified polyethylene glycol.

In one embodiment, the surface of the nanoparticle has modified terminal functional groups.

In one embodiment, the nanoparticle comprises a target structure.

In a preferred embodiment, the target structure is part of an antibody, ligands, aptamers, or a fragment thereof.

In addition, the object of this invention is achieved by a process for the production of a nanoparticle according to this invention, comprising an inclusion and charge complex according to this invention, whereby the process comprises the following steps:
(a) Dissolving a cationic active ingredient in a solvent;
(b) Bringing into contact the active ingredient with an anionic inclusion-forming agent;
(c) Producing an inclusion and charge complex while forming a nanoparticulate dispersion;
(d) Recovering the nanoparticle from the dispersion.

In one embodiment, the solvent in step (a) is an organic solvent, preferably methanol, ethanol or acetone.

In one embodiment, the dissolved active ingredient in step (b) is added to a solution that contains the inclusion-forming agent while being stirred continuously.

In one embodiment, the forming of the inclusion and charge complex in step (c) takes place over about 24 hours of stirring.

In one embodiment, a complete evaporation of the organic solvent takes place before the recovery of the nanoparticle in step (d).

In one embodiment, the recovery of the nanoparticle in step (d) comprises a separation of larger aggregates by filtering the nanoparticulate dispersion through a filter with a pore size of 1 μm.

In one embodiment, the recovery of the nanoparticle in step (d) comprises a concentration of the nanoparticulate suspension by ultrafiltration or vacuum rotary evaporation.

In one embodiment, the process for the production of a nanoparticle in addition comprises the following step:
(e) Freeze-drying the nanoparticulate suspension in the presence of cryoprotectors.

In one embodiment, the process for the production of a nanoparticle in addition comprises the following step:
(c′) Modifying the surface of the nanoparticle.

In a preferred embodiment, the modification in step (c′) consists in a formation of non-covalent electrostatic and/or covalent bonds.

In addition, the object of this invention is achieved by a use of a nanoparticle according to this invention for the production of a pharmaceutical preparation.

In one embodiment, the pharmaceutical preparation comprises a controlled release preparation.

In one embodiment, the pharmaceutical preparation comprises a pharmaceutical formulation that is insoluble in gastric juice, for example a capsule.

The term “active ingredient,” as used here, comprises therapeutically, diagnostically and cosmetically active compounds. Compounds that are active in animals other than humans and in plants are also included.

A “basic active ingredient,” as used here, comprises any basic active ingredient, preferably a weakly basic active ingredient. As basic active ingredients, all known cationic or basic active ingredients are considered. As salts of a basic active ingredient, for example, hydrochlorides, hydrobromides, sulfates, mesilates, malonates, tartrates and phosphates are considered. A “weakly basic active ingredient,” as used here, has a pK_{B} value=4.5-9.5 (weak base) or a pK_{B} value=9.5-14 (very weak base).

An “inclusion-forming agent,” as used here, is a component of the inclusion and charge complex and as such is a complex partner of the cationic active ingredient. This also applies for other molecules correspondingly mentioned here, such as, for example, proteins and peptides.

All anionically modified cyclodextrins cited above can be present in their basic structure as alpha-, beta-, gamma- or delta-cyclodextrin.

The term “meta-stable” means a state that is stable relative to small changes but is slightly unstable in the case of more significant changes. In connection with this invention, the changes relate to the presence of an “additional charged compound” or “another salt,” i.e., a compound or a salt that is not a component of the original inclusion and charge complex. By interaction with a physiological medium, e.g., the content of the gastrointestinal tract, and compounds or salts contained therein, the charge forces within the complex are weakened by interaction with external competing charges, which promotes the release of the active ingredient from the inclusion complex. In this case, the anionic inclusion-forming agent can pass through a recomplexing, i.e., it goes with the additional compound or the additional salt into new complexes. The previously complexed and enclosed active ingredient, supported by the establishment of diffusion equilibrium, is cleared of the original inclusion and charge complex or the newly formed complex from the inclusion-forming agents and additional compound or additional salt.

The term “surface potential,” also referred to as a surface charge, has the same meaning as the term “zeta-potential.”

A “modification of the surface” of a nanoparticle can be carried out by forming non-covalent or covalent bonds. A non-covalent modification of the negatively charged particle surface can be carried out by using electrostatic interactions with positively- or partially-positively charged compounds (charge titration). For surface modification, in this case, dipolar-dipolar interactions, van der Waals forces, hydrophobic interactions and hydrogen bridge bonds can also be used. A steric cross-linking of molecular areas of the surface-modifying substance is possible and has a stabilizing influence. The covalent bonds are formed by a chemical coupling reaction with a target structure or a stabilizing compound, whereby the reaction takes places between functional groups of the particle surface and the surface-modifying compound.

A “target structure” (target moiety) contains or consists of a structure that is able to interact with another structure at a site of action. By this property, the target structure makes possible a “targeting,” i.e., a targeting of the site of action, by which a nanoparticle can accumulate specifically at the site of action. The interaction can be mediated, for example, via receptors or special membrane proteins, which are enhanced or else are present exclusively in the target cells or in target tissues, e.g., tumor tissues. Such target structures comprise structures that mediate an active targeting and/or a passive targeting. The structures that mediate an active targeting include, for example, structures of an antibody, a receptor-ligand, ligand-mimetic agents or an aptamer. As structures, peptides, carbohydrates, lipids, nucleosides, nucleic acids, polysaccharides, modified polysaccharides or fragments thereof are considered. Target structures can also be transferrin or folic acid or portions thereof.

The term “controlled release” means that the active ingredient is released over time according to a specific pattern. This pattern can comprise a continuous or an intermittent release. A special form of a “controlled release” is a “sustained release,” which means that the active ingredient is released with a time delay in comparison to a conventional pharmaceutical formulation.

This invention discloses pharmaceutically useful nanoparticulate inclusion and charge complexes that are formed by inclusion and precipitation of (weakly) basic active ingredients in the protonated state with the adjuvant beta-cyclodextrin-phosphate. Hydrophobic molecular structures of the active ingredient with corresponding structural requirements are enclosed as guest complexes in the interior space of the beta-cyclodextrin phosphate.

The preferred embodiment of this invention thus makes it possible to convert both heavily and lightly water-soluble (weakly) basic active ingredients into a meta-stable nanoparticulate complex, whereby a combination that consists of two different mechanisms is used:
(1) Formation of a nanoparticulate inclusion complex; and
(2) Formation of a charge complex as a result of electrostatic interactions between the phosphate groups of anionically modified beta-cyclodextrins and the charges of the protonated pharmaceutical substance base.

The system that is carried by charges and hydrophobic interactions is stable in its meta-stable state over a broad pH range from 4 to 9 as a nanoparticle, with which a pure dissociation- and diffusion-controlled release of the pharmaceutical substance base from the particulate system in this pH range is possible, i.e., in the case of pH values that correspond to the pH values that are naturally present in the gastrointestinal tract. This release takes place independently of a polymer degradation and swelling processes of the particle components. The stability of the complex of these inventions is made possible by the special combination of the inclusion complex and electrostatic interactions. A stable particulate system is thus produced with only one adjuvant component, and said system releases its pharmaceutical substance that is defined by interactions with charged components of blood plasma or other physiological fluids via a special deaggregation behavior. Since the release of the pharmaceutical substance is largely independent of the pH, a uniform resorption is probable along the gastrointestinal tract.

The low viscosity of the beta-cyclodextrin solution makes possible the production of nanoparticles in a defined range of sizes. The additional use of a surfactant for stabilizing the nanoparticulate system is not necessarily required, since the system is stabilized electrostatically via the phosphate groups in the beta-cyclodextrin. Side effects that are produced by the use of a surfactant as an additional adjuvant can thus be avoided. On the other hand, the use of a surfactant also is not to be ruled out.

In addition, the stability and the deaggregation behavior of the nanoparticulate system can be modified by a surface modification with block co-polymers or target structures that are to improve the passive and active targeting.

Since a low water solubility of an active ingredient in general accompanies a low bioavailability after its administration in a pharmaceutical preparation, the nanoparticulate system of the invention also contributes to improving the bioavailability of sparingly water-soluble, (weakly) basic active ingredients.

Readily water-soluble salts of the basic active ingredients (e.g., hydrochlorides) can just as well be converted into such a nanoparticulate system. Here, the advantage lies in a specific concentration of the active ingredient at the site of action with use of passive and active targeting effects by the formulation as surface-modified nanoparticles.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German Application No. 102005041860.0, filed Sep. 2, 2005, and U.S. Provisional Application Ser. No. 60/713,332, filed Sep. 2, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### SPECIFIC DESCRIPTION OF THE INVENTION

The invention is to be explained in more detail below based on the examples together with the attached figures.

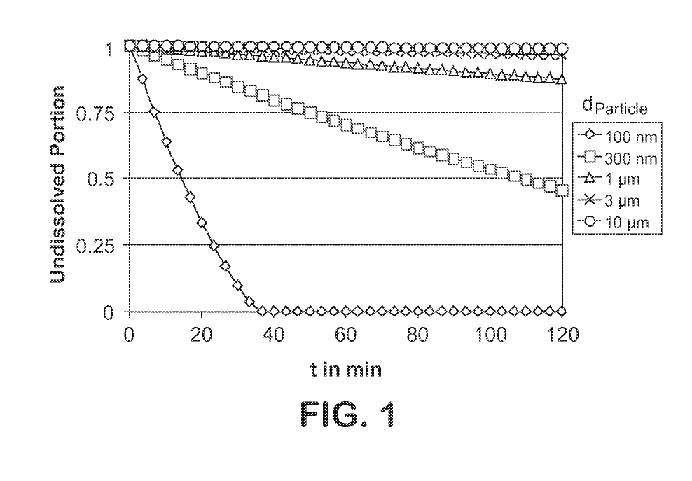

FIG. 1 illustrates the result of a model calculation of the time-dependent solubility of an active ingredient based on the diameter of a nanoparticle that contains the active ingredient. As a model active ingredient, vatalanib succinate is used.

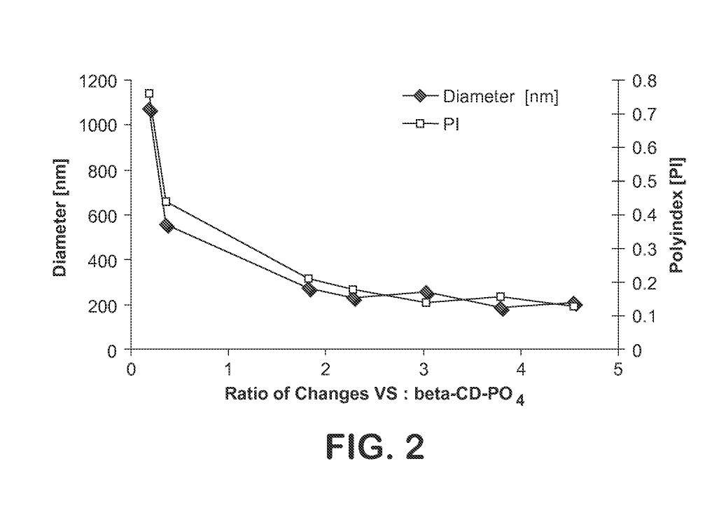

FIG. 2 shows the dependency of the particle size (diameter) as well as the size distribution (polydispersity index, PI) of nanoparticulate inclusion and charge complexes that consists of beta-cyclodextrin-phosphate (beta-CD-PO₄) and vatalanib succinate (VS) of the established charge ratio of the components.

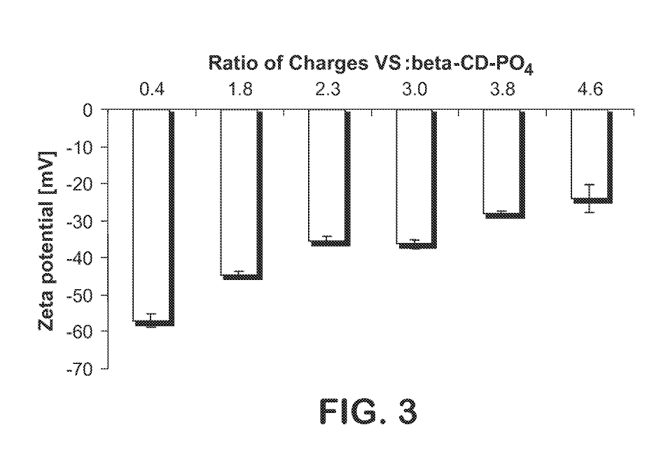

FIG. 3 shows the influence of the established charge ratio of vatalanib succinate (VS) and beta-cyclodextrin phosphate (beta-CD-PO₄) on the resulting surface potential, indicated as a zeta potential in [mV].

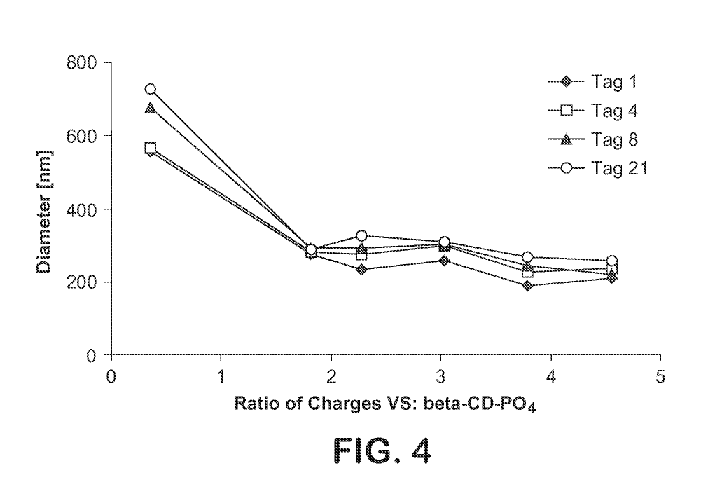

FIG. 4 shows the influence of the established charge ratio of vatalanib succinate (VS) and beta-cyclodextrin phosphate (beta-CD-PO₄) on the size stability of the nanoparticles after a period of one day up to three weeks.

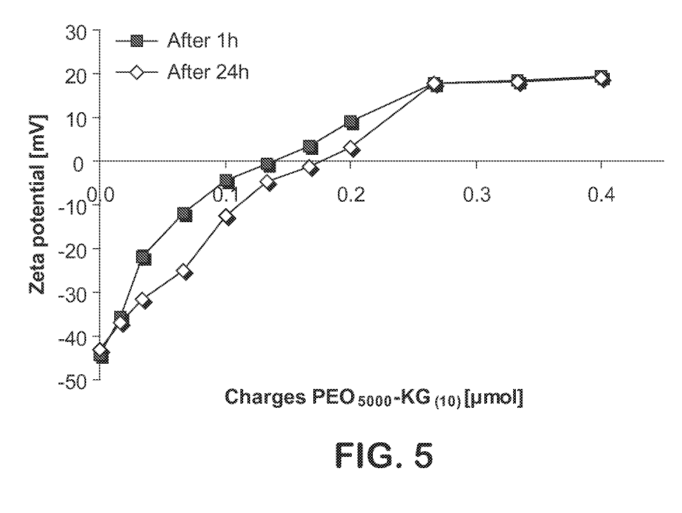

FIG. 5 shows the influence of a surface modification of by PEO₍₅₀₀₀₎-KG₍₁₀₎ on the zeta potential.

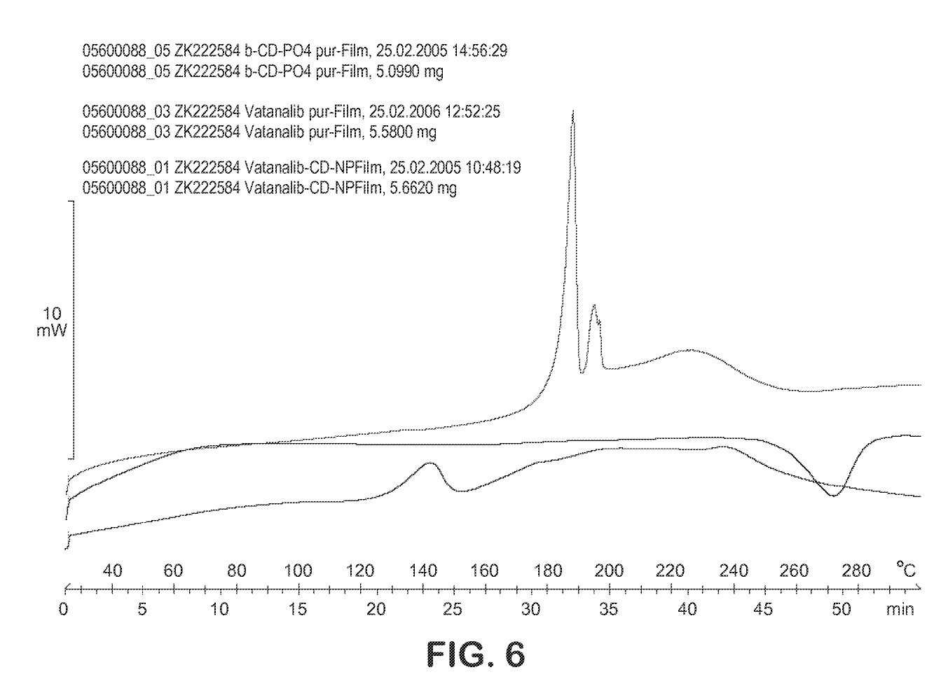

FIG. 6 shows the results of the DSC measurements of vatalanib succinate/beta-cyclodextrin-phosphate nanoparticles as well as uncomplexed vatalanib succinate (VS) and uncomplexed beta-cyclodextrin-phosphate as solids. The following arrangement is depicted (curves of the spectra viewed at the outside right edge):
Lower curve: Valatanib-succinate/beta-cyclodextrin-phosphate nanoparticles, dried, solid;
Middle curve: Beta-cyclodextrin phosphate, uncomplexed, solid;
Upper curve: Vatalanib succinate, uncomplexed, solid.

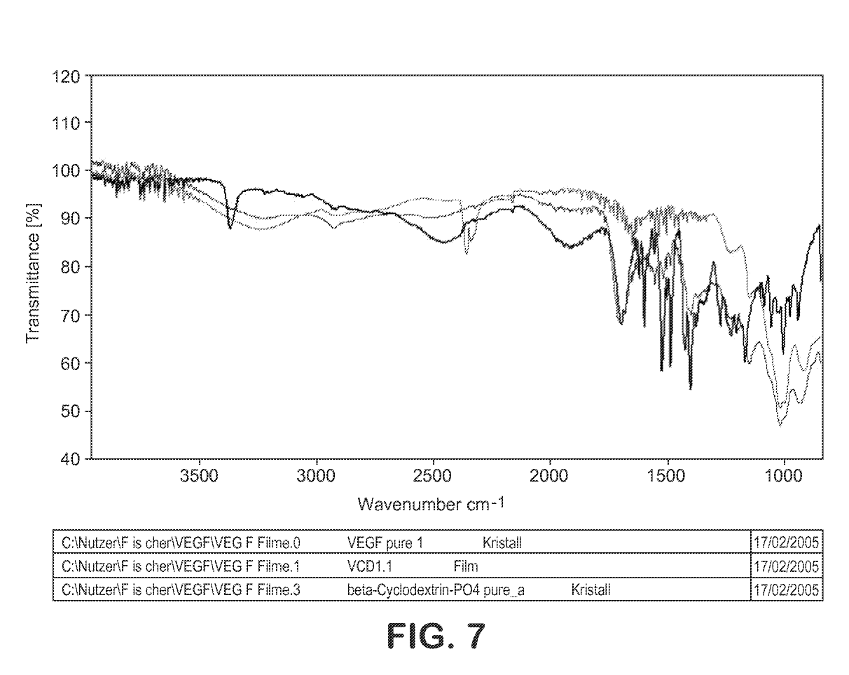

FIG. 7 shows the FT-IR spectra of vatalanib-succinate/beta-cyclodextrin-phosphate nanoparticles as well as uncomplexed vatalanib succinate and beta-cyclodextrin phosphate as solids. The following arrangement is depicted (curves of the spectra viewed at the outside right edge):
Upper curve: Valatanib succinate, uncomplexed, solid;
Middle curve: beta-cyclodextrin phosphate, uncomplexed, solid;
Lower curve: Vatalanib-succinate/beta-cyclodextrin-phosphate nanoparticles, dried, solid.

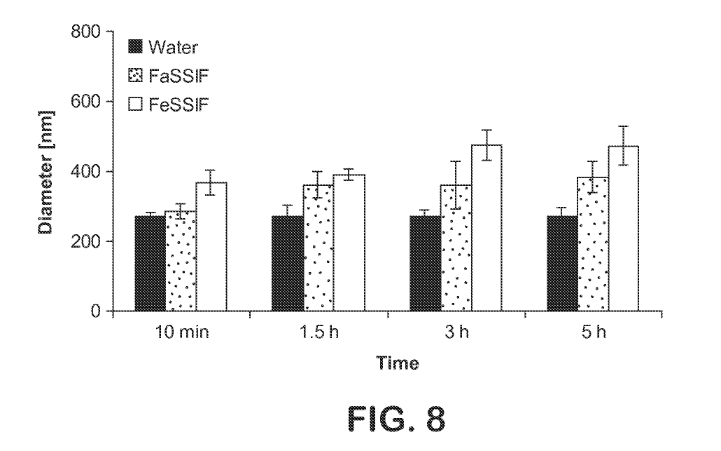

FIG. 8 shows the stability of vatalanib-succinate/beta-cyclodextrin-phosphate nanoparticles in two artificially recreated intestinal media, FaSSIF and FeSSIF, in comparison to water over a period of 5 hours.

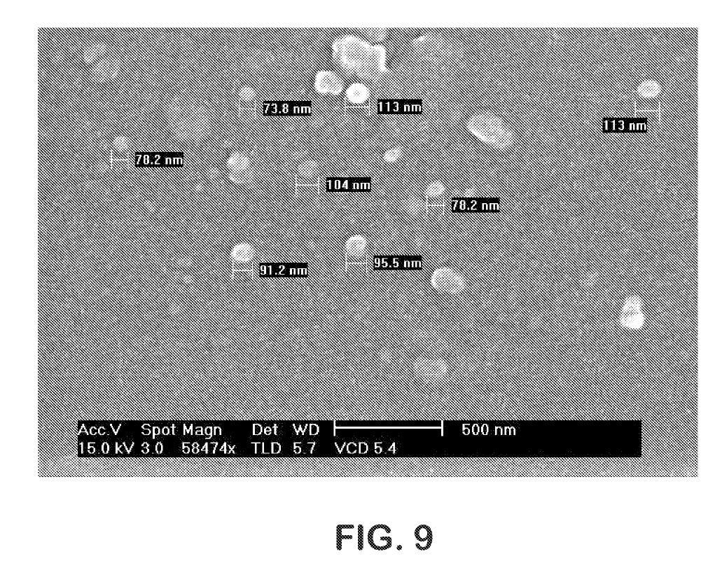

FIG. 9 shows REM images of spherical vatalanib-succinate/beta-cyclodextrin-phosphate nanoparticles with a particle size of about 100 nm.

### EXAMPLES

### Example 1

### Model Calculation of the Relationship Between the Proportion of Dissolved Active Ingredient and the Particle Size as a Function of Time in an Open System (Sink Conditions)

There is a connection between the dissolved proportion of active ingredient in an open system and its particle size. In the example of vatalanib succinate, FIG. 1 shows how this relationship is represented as a function of time. As a basis for the calculation, the Noyes-Whitney equation as well as different chemical-physical parameters, such as, e.g., the alteration of the particle surface, the alteration of the diameter, as well as the saturation solubility, were used.

The result that is shown in FIG. 1, according to which the undissolved proportion of active ingredient is all the more quickly reduced at a particle size of between 100 nm and 10 μm, the smaller the particles are, means that a smaller particle size accompanies an improved solubility of the active ingredient. Thus, in the case of a smaller particle size, free dissociated active ingredient is more quickly present, which is then available for resorption in the gastrointestinal tract. A result of this increased solubility is an improved bioavailability of the active ingredient.

### Example 2

### Measuring Process for Determining Particle Size

The size of the nanoparticles was determined with the aid of dynamic light scattering (Dynamic Light Scattering, DLS) with use of a “Zetasizer 3000” (Malvern Instruments). In addition, images in the Raster-electron microscope (REM) were made, as is shown by way of example in FIG. 9. FIG. 9 also confirms the spherical shape of the nanoparticles.

The determination of the particle size by DLS is based on the principle of photon correlation spectroscopy (Photon Correlation Spectroscopy, PCS). This process is suitable for measuring particles with a size in the range of 3 nm to 3 μm. The particles, in solution, are subjected to an undirected movement, triggered by the collision with liquid molecules of the dispersing agent, whose driving force is Brown's molecular movement. The resulting movement of the particles is all the faster the smaller their particle diameter is. If a sample is irradiated in a cuvette with laser light, it results in the particles moving in an undirected manner for scattering light. By this movement of particles, the scattering is not constant, but rather fluctuates over time. The fluctuations of the intensity of the scattered laser light, detected at a 90° angle, are all the greater the faster the particles move, i.e., the smaller they are. On the basis of these intensity fluctuations, the particle size can be derived with the aid of an auto-correlation function. The mean particle diameter is calculated from the drop in the correlation function. For correct calculation of the average particle diameter, the particles should have a spherical shape, which can be examined by REM images (see above), and the particles should not settle out or float. The measurements were made with samples in suitable dilution under a constant temperature of 25° C. as well as a defined viscosity of the solution.

### Example 3

### Measuring Process for Determining the Surface Potential

The surface potential, also referred to as the zeta potential, indicates the potential of a migrating particle at the shear plane, i.e., if the majority of the diffuse layer has been sheared off by the movement of the particle. The surface potential was determined with the process of laser-Doppler anemometry with use of a “Zetasizer 3000” (Malvern Instruments).

Particles with a charged surface migrate into an electrical field for oppositely charged electrodes, whereby the migrating speed of the particles depends on the amount of surface charges and the applied field strength. The thus mentioned electrophoretic mobility is produced from the quotient of the migrating speed and the electric field strength. The product that consists of the electrophoretic mobility and the factor 13 corresponds to the zeta potential whose unit is [mV].

The laser-Doppler anemometry method determines the migrating speed of the particles in the electric field. To this end, particles that migrate into the electric field are irradiated with a laser, and the scattered laser light is detected. By the movement of the particles, a frequency displacement with reflected light is measured in comparison to irradiated light. The distance of this frequency displacement depends on the migrating speed and is referred to as the thus-mentioned Doppler frequency (Doppler effect). The migrating speed of a particle can be derived from the Doppler frequency, the scattering angle and the wavelength.

### Example 4

### Production of Vatalanib-Succinate/beta-Cyclodextrin-Phosphate Nanoparticles

A 1.37% methanolic solution of vatalanib succinate was quickly undersprayed by a 0.1% beta-cyclodextrin-phosphate solution (Fluka, CAS No. 199684-61-2) while being stirred constantly. The batch was stirred for about 24 hours and then filtered through a spray filter with a pore size of 0.1 μm. The hydrodynamic diameter of the samples was determined by means of DLS (see the example above). The ratio of the charge moles that were used was decisive for the particle size and the size distribution of the nanoparticles. It can be seen from FIG. 2 that by using excess vatalanib succinate, smaller and more stable particles of around 200 nm were formed with a narrow particle size distribution. This is reflected in a decrease of the polydispersity index by about 0.7 to about 0.1 in a ratio of charges of >1:1. The polydispersity index is a measurement of the breadth of the distribution of the particle size, whereby higher values indicate a broader distribution. For the polydispersity index, values of between 0 and 1 are indicated by the measuring device, whereby values of between 0.5 and 1 are to be evaluated critically. A monomodal distribution was present in the measured samples.

### Example 5

### Stability of Vatalanib-Succinate/Beta-Cyclodextrin-Phosphate Nanoparticles

FIG. 3 shows the result of determining the surface-associated charges of the samples, composed in different ways, after three weeks. The zeta potential, determined via the measurement of the electrophoretic mobility at a constant pH, was in the negative range of between −20 to −60 mV in all samples. Consequently, all particle samples, independently of the charge ratio between vatalanib succinate and beta-cyclodextrin phosphate used for production, were electrostatically stabilized via the phosphate groups found in the beta-cyclodextrin.

FIG. 4 confirms the stability of the particles in aqueous solution over a period of 3 weeks. Larger aggregates showed the tendency toward particle growth, while the stable and narrowly distributed particle samples starting from a charge ratio of about 1:1 were constant in size.

### Example 6

### Influence of Surface Modifications

FIG. 5 shows the change in surface-associated charges (zeta potential) by modification of the particle surface with a 0.1% solution of the block co-polymer PEG₍₅₀₀₀₎-KG₍₁₀₎ (PEG=polyethylene glycol=polyethylene oxide; K=arginine; G=glycine). The initial sample with a zeta potential of about −45 mV was mixed with increasing amounts of the block co-polymer PEO₍₅₀₀₀₎-KG₍₁₀₎. This led to a compensation of excess negative surface charges of the phosphate groups, detectable in a steadily increasing zeto potential. The compensation of the charges was carried out above the neutral point of 0 mV up to a dissociation equilibrium at a zeta potential of about +20 mV. Another addition of the block co-polymer PEO₍₅₀₀₀₎-KG₍₁₀₎ did not show any further increase of the surface potential. Thus, the particle surface was successfully modified by electrostatic interactions between the negatively charged phosphate groups of the cyclodextrin, stabilizing the surface, and the cationically charged block of the block co-polymer PEO₍₅₀₀₀₎-KG₍₁₀₎. The polyethylene (PEG) block that covers the particle surface can make a contribution to increasing the stability of the particle by means of additional steric stabilization, and in the case of an intravenous application, it can produce an extended circulation in the blood flow. A reason is the shielding of the particle from opsonizing proteins and thus the protection from a quick uptake by macrophages with subsequent degradation in the reticuloendothelial system. This corresponds to the principle of stealth liposomes.

### Example 7

### Comparison of the Properties of Vatalanib Succinate and Beta-Cyclodextrin Phosphate in the Uncomplexed and Complexed State

FIG. 6 shows the results of Differential Scanning Calorimetry (DSC) measurements. Vatalanib succinate has a melting point at a temperature of 190-200° C., which is characteristic of the crystalline state of the substance. The curve of the pure beta-cyclodextrin phosphate has a decomposition peak at about 275° C. The DSC curve that results from the complex has a thermal transition at about 140° C. In the range of the melting temperature of vatalanib succinate, the DSC curve of the complex does not show any peak, from which the absence of crystalline and thus unbonded vatalanib succinate can be deduced. The absence of vatalanib succinate in the complex and the thermal transition of the complex at 140° C., presumably the melting point of the complex, are references to a strong interaction between vatalanib succinate and beta-cyclodextrin phosphate, based on the formation of a charge and inclusion complex.

The FT-IR (Fourier Transformation/infrared) spectra shown in FIG. 7 support this finding. FIG. 7 shows the FT-IR spectra of pure vatalanib succinate, pure beta-cyclodextrin phosphate as well as the complex that consists of vatalanib succinate and beta-cyclodextrin phosphate. Characteristic of the spectrum of vatalanib succinate is the sharp peak at 3300 nm, which results from the oscillation of R2-NH. The number of aromatic rings in the vatalanib-succinate molecule is responsible for the strong oscillations in the so-called fingerprint area. In contrast to this, the beta-cyclodextrin phosphate, which does not have any aromatic system, has hardly any or very poorly pronounced oscillations in the fingerprint area. The spectrum of the complex is distinguished in that the R2-NH peak that is characteristic of vatalanib succinate disappears. This can be explained with the formation of a charge complex between protonated R2-NH2+ and the phosphate groups of the beta-cyclodextrin. In the spectrum of the complex, the fingerprint area is covered mainly with that of the pure beta-cyclodextrin phosphate, which indicates the formation of inclusion complexes of the aromatic molecule structures of vatalanib succinate, and as a result thereof, the strongly aromatic oscillations are suppressed in the fingerprint area and thus do not appear in the spectrum of the complex.

### Example 8

### Stability of Vatalanib-Succinate/beta-Cyclodextrin-Phosphate Nanoparticles under Physiological Conditions

To simulate the stability of nanoparticles in the case of an oral administration in the gastrointestinal tract, the particles were tested in biorelevant media.

To this end, time-dependent particle size studies of vatalanib-succinate/beta-cyclodextrin-phosphate nanoparticles in FaSSIF (Fasted State Simulated Fluid) and FeSSIF (Fed State Simulated Fluid) were performed in comparison to water. FaSSIF and FeSSIF are the above-mentioned biorelevant media, which simulate the situation in vivo by their composition:
FaSSIF: Fluid in the proximal small intestine in the empty state with respect to the pH (pH 6.5), osmolality and concentration of the gallbladder components.
FeSSIF: Fluid in the proximal small intestine in the postprandial state (after mealtime) with respect to the pH (pH 5), osmolality, concentration of the gallbladder components.

FIG. 8 shows the results of these measurements. In a period of 5 hours, the particles that are incubated in FaSSIS and FeSSIF show a minimum particle growth. Altogether, the particle sizes remain stable in the nanometer range, and formation of larger aggregates or precipitation of the particles does not result.

### Example 9

### Nanoparticles that Consist of Imipramine Hydrochloride and Beta-Cyclodextrin Phosphate

A 1% aqueous solution of imipramine hydrochloride (Sigma, CAS No.: 113-52-0) was quickly undersprayed by a 0.1% beta-cyclodextrin-phosphate solution (Fluka, CAS No. 199684-61-2) while being stirred constantly. The batch was stirred for about 24 hours. The particle size can be controlled via the established charge ratio. Stabilization was carried out by an excess of negative charges of the phosphate groups.

Apomorphine hydrochloride and beta-cyclodextrin phosphate also can be used accordingly to produce nanoparticles.

### LITERATURE

¹WO 98/35958
²Lahr et al.; U.S. Pat. No. 5,368,864;
³Kanikanti et al.; U.S. Pat. No. 5,707,655;
⁴Nakamichi et al.; U.S. Pat. No. 5,456,923;
⁵Bibby, D., Davie, N. M., Tucker, I. G. (2000), Mechanisms by Which Cyclodextrins Modify Drug Release from Polymeric Drug Delivery Systems; Int J Pharm 197: 1-11;
⁶Rowe, R. C., Sheskey, P. J., Weller, P. J., Handbook of Pharmaceutical Excipients, Fourth Edition: 186-190;
⁷Bellocq, N. C., Pun, S. H., Jensen, G. S., Davis, M. E. (2003), Transferrin-Containing Cyclodextrin Polymer-Based Particles for Tumor-Targeted Gene Delivery, Bioconjug Chem. 14: 1122-1132;
8Pun, S. H., Tack, F., Bellocq, N. C., Cheng, J., Grubbs, B. H., Jensen, G. S., Davis, M. E., Brewster, M., Janicot, M., Janssens, B., Floren, W., Bakker, A. (2004), Targeted Delivery of RNA Cleaving DNA Enzyme to Tumor Tissue by Transferrin-Modified, Cyclodextrin-Based Particles; Cancer Biol Ther 3: 641-650;
⁹WO 03/072367
¹⁰WO 02/49676
¹¹WO 00/41704
¹²Duchene, D., Wouessidjewe, D., Ponchel, G. (1999), Cyclodextrins and Carrier Systems; J Control Release 62: 263-268

1. Nanoparticulate inclusion and charge complex, comprising at least two complex partners, whereby a complex partner is an anionic inclusion-forming agent and another complex partner is a cationic active ingredient.

2. Nanoparticulate inclusion and charge complex according to the claim, whereby the cationic active ingredient is a basic active ingredient.

3. Nanoparticulate inclusion and charge complex according to claim 1, whereby the basic active ingredient is in the protonated state.

4. Nanoparticulate inclusion and charge complex according to claim 1, whereby the cationic active ingredient is a low-molecular active ingredient.

5. Nanoparticulate inclusion and charge complex according to claim 1, whereby the inclusion-forming agent is an anionically modified cyclodextrin.

6. Nanoparticulate inclusion and charge complex according to claim 5, whereby the anionically modified cyclodextrin is selected from the group that consists of cyclodextrin phosphate, cyclodextrin sulfate, cyclodextrin carboxylate and cyclodextrin succinate.

7. Nanoparticulate inclusion and charge complex according to claim 5, whereby the anionically modified cyclodextrin is a beta-cyclodextrin phosphate.

8. Nanoparticulate inclusion and charge complex according to claim 5, whereby the anionically modified cyclodextrin is heptakis-(2,3-dimethyl-6-sulfato)-beta-cyclodextrin or heptakis-(2,6-diacetyl-6-sulfato)-beta-cyclodextrin.

9. Nanoparticulate inclusion and charge complex claim 1, whereby the active ingredient is selected from the group that consists of pynalin, vatalanib succinate, imipramine, apomorphine, atropine, scopolamine, bamipine, astemizole, diphenhydramine, quinidine, quinine, chloroquine, chlorpromazine, chlorprothixene, codeine, ephedrine, naphazoline, oxedrine, isoprenaline, salbutamol, fenoterol, hydromorphone, hydrocodone, morphine, haloperidol, imipramine, lidocaine, loperamide, methadone, levomethadone, metoclopramide, cimetidine, naphazoline, perazine, pethidine, procaine, benzocaine, lidocaine, mepivacaine, promazine, chlorpromazine, propanolol, scopolamine, perazine, thioridazine, trimethoprim, bromhexine, clotrimazole, nitroflurantoin, diazepam, oxazepam, nitrazepam, diphenhydramine, haloperidol, imipramine, isoniazid, loperamide, metronidazole, nicotinamide, papaverine, pethidine, phenazone, ambroxol, bamipine, diphenhydramine, bromocriptine, clonidine, propanolol, metoprolol, phentolamine, sulfaguanidine, ergotamine, verapamil, diltiazem, neostigmine bromide, pilocarpine, physostigmine, ketotifen, thiamin, pyridoxine, imiquimod, irinotecan, raloxifene, tirofiban, mercaptamine bitartrate, brimonidine, tolterodine, mizolastine, abacavir, zaleplon, emedastine, amisulpride, sibutramine, levacetylmethadol, rizatriptan, lercandipine, rosiglitazon, buproprion, quetiapin, brinzolamide, lomefloxacin, almotriptan, galanthamine, desloratadine, levocetirizine, levodropropizine, oxaprozin, voriconazole, tiotropium bromide, ziprasidone, ebastine, eletriptan, imantinib, gatifloxacin, olmesartan, frovatriptan, solifenacin, manidipine, epinastine, olopatadine, escitalopram, duloxetine, a therapeutically active protein, a therapeutically active peptide, and salts thereof.

10. Nanoparticulate inclusion and charge complex according to claim 9, whereby the active ingredient is vatalanib succinate.

11. Nanoparticulate inclusion and charge complex according to claim 1, whereby the complex is meta-stable.

12. Nanoparticulate inclusion and charge complex according to claim 1, whereby the complex dissociates from inclusion-forming agents and active ingredients in the presence of another charged compound or another salt.

13. Nanoparticulate inclusion and charge complex according to claim 12, whereby the additional compound or the additional salt is contained endogenically in the gastrointestinal tract and/or is fed exogenically.

14. Nanoparticulate inclusion and charge complex according to claim 12, whereby the inclusion-forming agent and the additional charged compound or the additional salt accompany a complex and the dissociated active ingredient diffuses.

15. Nanoparticulate inclusion and charge complex according to claim 1, whereby in the range of pH 4 to pH 9, the stability of the complex is independent of pH.

16. Nanoparticulate inclusion and charge complex according to claim 1, whereby in the range of pH 5 to pH 7.5, the stability of the complex is independent of pH.

17. Nanoparticulate inclusion and charge complex according to claim 1, whereby the complex is stable in a simulated intestinal fluid, selected from FaSSIF and FeSSIF.

18. Nanoparticle comprising an inclusion and charge complex according to claim 1.

19. Nanoparticle according to claim 18, which comprises a surface modifying the inclusion and charge complex.

20. Nanoparticle according to claim 18, which has a size in the range of 10 nm to 1.2 μm.

21. Nanoparticle according to claim 20, which has a size in the range of 10 nm to 500 nm.

22. Nanoparticle according to claim 21, which has a size in the range of 10 nm to 300 nm.

23. Nanoparticle according to claim 1, whereby the surface has a negative surface potential in the range of −10 mV to −70 mV.

24. Nanoparticle according to claim 23, whereby the surface has a negative surface potential in the range of −20 mV to −60 mV.

25. Nanoparticle according to claim 1, which comprises at least one compound that modifies the surface.

26. Nanoparticle according to claim 25, whereby the compound that modifies the surface is covalently bonded or non-covalently-bonded to the surface of the nanoparticle.

27. Nanoparticle according to claim 25, whereby the compound that modifies the surface has a charge that is opposite to the charge of the surface of the nanoparticle.

28. Nanoparticle according to claim 25, whereby the compound that modifies the surface is a positively charged compound.

29. Nanoparticle according to claim 28, whereby the positively charged compound is a block co-polymer.

30. Nanoparticle according to claim 29, whereby the block co-polymer is a cationically modified polyethylene glycol.

31. Nanoparticle according to claim 18, whereby the surface has modified terminal functional groups.

32. Nanoparticle according to claim 18, which comprises a target structure.

33. Nanoparticle according to claim 32, whereby the target structure is a part of an antibody, ligands, aptamers or a fragment thereof.

34. Process for the production of a nanoparticle according to claim 18, comprising an inclusion and charge complex comprising at least two complex partners, whereby a complex partner is an anionic inclusion-forming agent and another complex partner is a cationic active ingredient, whereby the process comprises the following steps:
(a) Dissolving a cationic active ingredient in a solvent;
(b) Bringing the active ingredient into contact with an anionic inclusion-forming agent;
(c) Producing an inclusion and charge complex with the formation of a nanoparticulate dispersion;
(d) Recovering the nanoparticle from the dispersion.

35. Process according to claim 34, whereby the process, in addition, comprises the following step:
(c′) Modifying the surface of the nanoparticle.

36. Process according to claim 35, whereby the modification in step (c′) is a formation of non-covalent electrostatic and/or covalent bonds.

37. Use of a nanoparticle according to claim 18 for the production of a pharmaceutical preparation.

38. Use according to claim 37, whereby the pharmaceutical preparation comprises a controlled-release preparation.

39. Use according to claim 37, whereby the pharmaceutical preparation comprises a formulation that is insoluble in gastric juice.
